(19) United States
(12) Patent Application Publication
(10) Pub. No.: US 2003/0105505 A1
(43) Pub. Date: Jun. 5, 2003

(54) Medical leads with superior handling characteristics

(76) Inventor: Anne M. Pianca, Valencia, CA (US)

Correspondence Address: ADVANCED BIONICS CORPORATION, 12740 SAN FERNANDO ROAD, SYLMAR, CA, 91342, US

(21) Appl. No.: 10/305,924

(22) Filed: Nov. 26, 2002

Related U.S. Application Data
(60) Provisional application No. 60/338,248, filed on Dec. 5, 2001.

(51) Int. Cl. A61N 1/05

(52) U.S. Cl. 607/122

(57) ABSTRACT

A lead with at least one internal lumen is provided with superior slidability properties between the internal lumen and an inserted device (e.g., recording microelectrode, stylet). The at least one lumen may be lined with a material such as Teflon®.

The present application claims the benefit of U.S. Provisional Patent Application Serial No. 60/338,248, filed Dec. 5, 2001, which application is incorporated herein by reference in its entirety.

### FIELD OF THE INVENTION

The present invention relates to implantable medical leads, and more particularly to implantable medical leads with superior handling characteristics.

### BACKGROUND OF THE INVENTION

Leads are used in multiple medical situations. In many instances, the lead includes one or more internal lumens through which various items, such as stylets, microelectrodes, and other wires or wire-like devices, may slide. The ability of these inserted items to slide freely through the lead and/or for the lead to slide freely over the inserted items is important in many medical lead uses.

### SUMMARY OF THE INVENTION

The present invention addresses the above and other needs by providing improved leads and methods for improving the ability of various items, such as stylets, microelectrodes, wires, and wire-like devices, to slide through internal lumens of medical leads and/or for the leads to slide over such items.

### BRIEF DESCRIPTION OF THE DRAWINGS

The above and other aspects of the present invention will be more apparent from the following more particular description thereof, presented in conjunction with the following drawings wherein:

FIG. 1 is a side view of a lead comprising an electrode array and an offset;

FIG. 2 is a detail view of the offset portion of the lead of FIG. 1;

FIG. 3 is a detail view of the electrode array portion of the lead of FIG. 1;

FIG. 4A is a cross-section view of a control lead taken along line 4A-4A of FIG. 2;

FIG. 4B is a cross-section view of a control lead taken along line 4B-4B of FIG. 2;

FIG. 4C is a cross-section view of a control lead taken along line 4C-4C of FIG. 3;

FIG. 5A is a cross-section view of a lead of the present invention taken along line 5A-5A of FIG. 2;

FIG. 5B is a cross-section view of a lead of the present invention taken along line 5B-5B of FIG. 2; and

FIG. 5C is a cross-section view of a lead of the present invention taken along line 5C-5C of FIG. 3.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

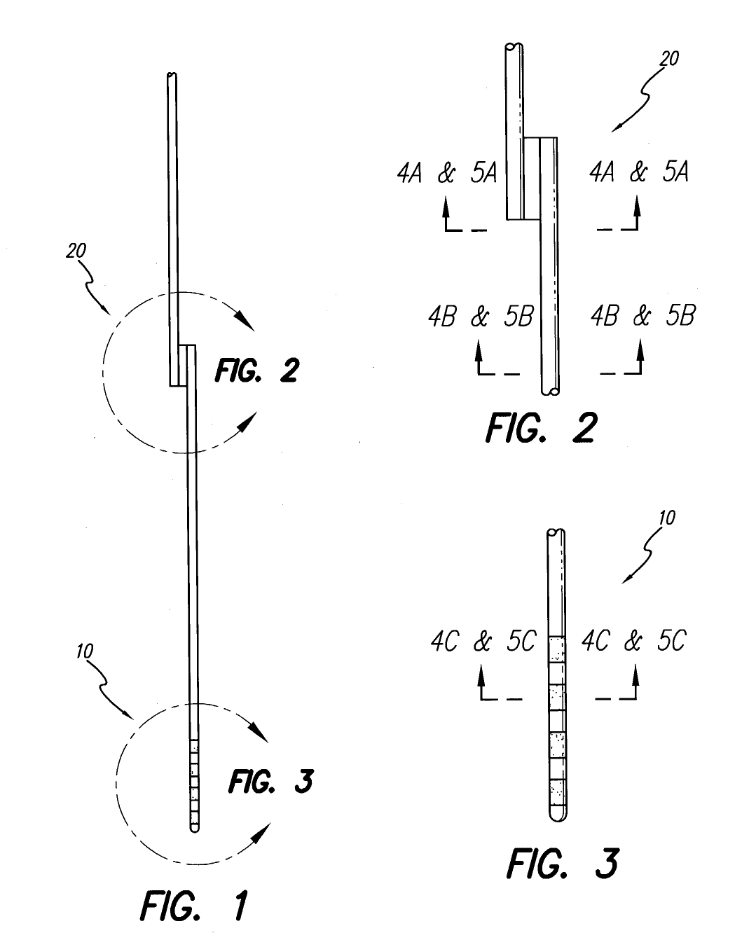

For purposes of describing the leads of the present invention, a comparison will be made with “control” leads. In addition, the leads tested were Deep Brain Stimulation (DBS) leads, although the utility of the invention is not limited to DBS leads. The control leads and the leads of the present invention comprise an electrode array 10 and offset 20, as shown in FIGS. 1, 2, and 3, although an offset is not critical to the invention. In addition, the leads tested and described contain one lumen 30, although the invention may be extended to work with leads including multiple lumens.

Control leads were constructed, including molded electrode array 10 and offset 20. Silicone was used for the molded sections of these control leads; however, other insulating material(s) (e.g., polyurethane) may be used. To evaluate performance, the control leads were tested with recording microelectrodes. Friction between the microelectrode and inner lumen 30 of the control leads, and in particular, the molded sections of the control leads, severely limited independent movement of both the microelectrode and the DBS lead. Difficulty sliding the microelectrode through the DBS lead is undesirable for the following reasons:

1. The microelectrode is typically advanced to the target DBS site to verify correct neuronal firing signals.

2. To verify stimulation efficacy, the DBS lead is typically advanced over the microelectrode to the target site that has been located with the microelectrode.

3. When the microelectrode is removed from the lead, it should not disturb the location of the indwelling DBS electrodes.

An effort was made to improve the “slidability” of the microelectrode and lead, while attempting to minimize changes to the control lead design and while maintaining an overall working diameter of the lead at a maximum of 1.1 mm (although the present invention is not limited to leads of this diameter). Therefore, a second iteration of DBS leads was built; these leads were made with larger internal lumens. Although sliding of the recording microelectrode through the lumen of these leads was improved, it was also deemed unacceptable. Next, a polytetrafluoroethylene (a.k.a., PTFE, e.g., Teflon®, made by E. I. du Pont de Nemours and Company of Wilmington, Del.) coated wire with the same overall diameter as the recording microelectrode was evaluated with the larger lumen lead. Once again, this provided only minuscule improvement.

The coefficient of static friction between steel and Teflon is approximately 0.04. The coefficient of static friction between steel and silicone is approximately 0.40. Thus, it was hypothesized that a DBS lead with a Teflon liner placed in the inner lumen of the lead would reduce the friction encountered between the inner lumen of the DBS lead and inserted wire. This hypothesis was tested and the results are shown below in Table 1.

| | Maximum | Maximum |
|---|---|---|
| | Resistance through | Resistance through |
| | DBS Lead WITHOUT | DBS Lead WITH |
| Wire Configuration | Teflon Liner (grams) | Teflon Liner (grams) |
| 0.015 inch diameter | 145 | 50 |
| 304 SS | | |
| 0.012 inch diameter | 15 | <5 |
| Teflon coated 304 SS | | |

Table 1 shows the maximum resistance encountered when advancing each of two different wire configurations through two different DBS leads. The diameter of the lumen in each lead was identical, but one lumen had a Teflon liner and the other did not.

As shown in Table 1, the maximum resistance encountered when advancing an uncoated 0.015 inch diameter wire made of 304 stainless steel through a DBS lead without a Teflon liner was approximately three times greater than what was encountered with the lead with a Teflon liner. Note also that, in the test of the uncoated wire inserted into the lead without Teflon liner, the wire jammed in the distal molded section (i.e., at electrode array 10), causing the lead to stretch, and the test was stopped in order to preserve the integrity of the lead.

As can also be seen in Table 1, the maximum resistance encountered with either lead was less for a smaller diameter Teflon coated wire than the uncoated wire. However the maximum resistance encountered by the Teflon coated wire was still significantly greater when passing the coated wire through the lead without a Teflon liner, compared with passing the coated wire through the lead with the Teflon liner.

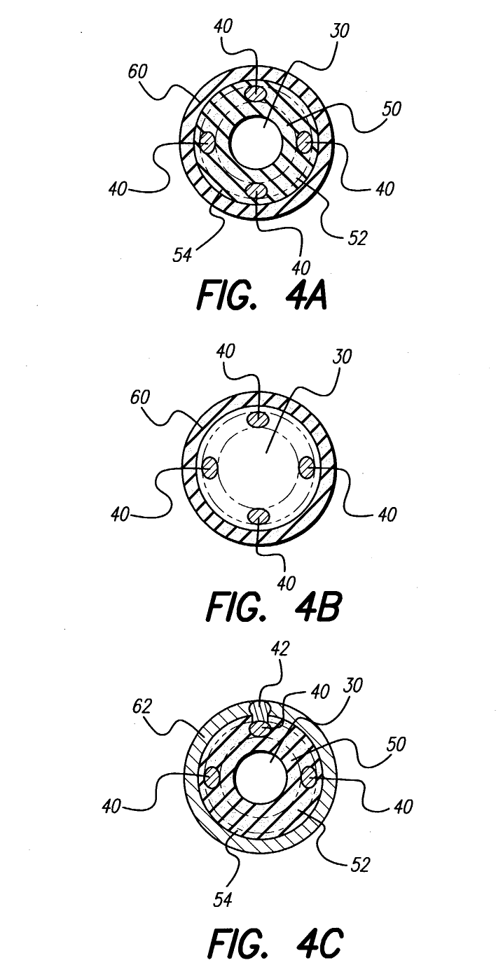

The reason for the excessive resistance encountered when placing a wire through a lead without a Teflon liner is described in relation to FIGS. 4A, 4B, and 4C. FIGS. 4A, 4B, and 4C are cross-sectional views of a lead as shown in FIGS. 1, 2, and 3, when such lead is without a liner. In the molded regions of the lead (e.g., in FIGS. 4A and 4C at cross-sections 4A-4A and 4C-4C), the mold material (e.g., silicone, polyurethane, or other suitable insulating material) flows around conductor winding 40 in coil region 52, fills any gaps between winding 40 and outer insulation 60 (FIG. 4A) or electrode 62 (FIG. 4C) in outer overflow region 54, and encroaches into inner lumen 30 in lumen overflow region 50. As a result, inner lumen 30 is partially obstructed by lumen overflow material 50. This is not the case for the non-molded section of the lead, e.g., FIG. 4B at cross-section 4B-4B. As a result of the high coefficient of friction of, e.g., silicone, the molded regions cause the wire to “jam”. The friction encountered is significantly increased when the wire is advanced through both molded sections 10 and 20 (e.g., when advancing the microelectrode to the target site prior to advancing the lead).

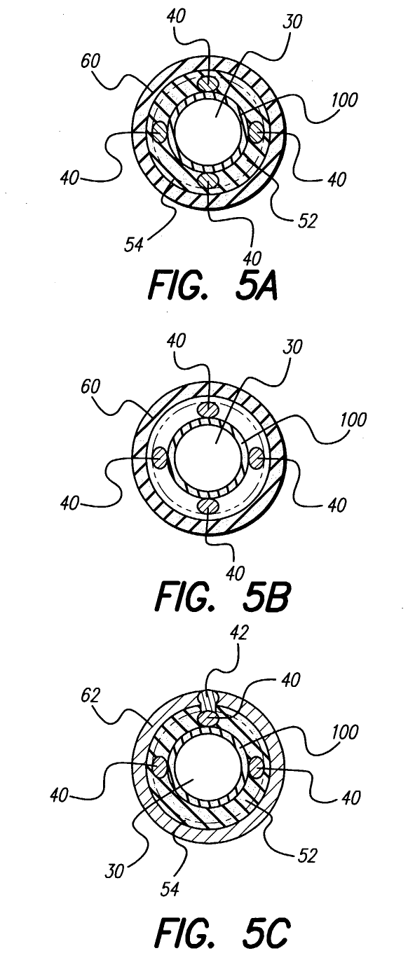

FIGS. 5A, 5B, and 5C are cross-sectional views of a lead as shown in FIG. 1, when such lead includes a liner 100 of Teflon® or the like material (e.g., fluorinated ethylene propylene (a.k.a., PEF), polyurethane, polyester, polyimide) with a preferred coefficient of friction with steel of about 0.20 or less, or even less than about 0.05. As can be seen by comparing FIGS. 4A and 5A or FIGS. 4C and 5C, liner 100 prevents the overflow of material 50 into the lumen 30 that is used for wire passage. In other words, the mold material used in over-molding electrode array 10 and offset 20 still flows around conductor winding 40 in coil region 52, and still fills any gaps between winding 40 and outer insulation 60 (FIG. 5A) and any gaps between winding 40/weld 42 and electrode 62 (FIG. 5C), but the material is prevented by liner 100 from encroaching into inner lumen 30. Thus, in FIGS. 5A and 5C, there is no lumen overflow region 50. Due to the extremely low coefficient of friction of Teflon, the wire slides with little, and almost no, resistance through the entire lumen 30 of the lead.

In addition to having a very low coefficient of friction, Teflon elongates very little under an axial load, and in tube form, has a uniform inner and outer diameter. This is an additional advantage for the DBS lead, as will now be explained. Target stimulation sites are located with micron precision. A lead with an inner liner 100 that essentially does not elongate, such as a liner 100 made of Teflon, means the lead will be less likely to stretch and create a source of error in the longitudinal direction. This is superior to, for example, a silicone lead without a Teflon liner, since silicone has a higher percent elongation (780-810%) than Teflon (200-400%).

Another advantage of a Teflon liner is that it makes the walls of lumen 30 uniform and free of voids. This minimizes the possibility of an inserted wire protruding through, e.g., conductor winding 40, and puncturing the outer insulation 60 of the lead. This is especially useful during the step of advancing a recording microelectrode through the inner lumen 30 of the lead, since recording microelectrodes have small, needle-sharp tips that can easily migrate through any space in conductor winding 40 and protrude through outer insulation 60.

A lead of the present invention may be constructed, for instance, according to the following procedure:

1. Slide a tube of Teflon, or other suitable liner 100 material, over a mandrel. Liner 100 may be etched along some or all of its length. For instance, it may be etched only where over-molding will occur. (Etched Teflon tubing may be purchased, or smooth Teflon tubing may be etched using techniques familiar to those of skill in the etching arts.) Such etching may improve cohesion of liner 100 to, for instance, a material used for over-molding and/or a material used for outer insulation 60.

2. Wind a conductor(s), such as a wire, cable, insulated wires or cables, or the like, around liner 100. Alternatively, slide a pre-wound coil over the mandrel. In the examples herein, four insulated wires make up conductor winding 40, although any number of wires or cables may be used.

3. Slide a tube of outer insulation 60 over the conductor(s), e.g., winding 40. Outer insulation 60 may be a tube made of silicone, polyurethane, or the like. Further, outer insulation 60 and/or any other insulation used in the lead assembly (e.g., insulation on wire or cables of conductor winding 40) may be made of the liner 100 material (e.g., Teflon®). For instance, conductors (e.g., winding 40) embedded in Teflon may be the liner.

4. Electrically connect the conductor(s) to electrode(s) 62 (e.g., each of four electrodes of electrode array 10 is electrically connected to a respective one of four conductors of conductor winding 40) via welding and/or other methods known in the art.

5. Place assembly in a mold or molds to over-mold the areas at the electrode array 10 and offset 20. The mold material is preferably, but not necessarily, the same material as was chosen for outer insulation 60, such as silicone, polyurethane, or the like. The over-molding process is known to those of skill in the art.

6. Any remaining construction and/or testing steps are performed as is traditional/desired.

In some embodiments, wire or cable conductor(s) may not be coiled into a conductor winding 40. In such instances, the conductors may be embedded in (e.g., surrounded by, positioned between or in) an insulation tube(s) such as silicone, polyurethane, liner material, or the like, which tube(s) may surround (or be) liner 100. It is also an option to embed coiled conductor(s) in a tube(s). Alternatively, the conductors may be positioned in lumens of a multi-lumen tube. For example, each wire or cable conductor may be placed in a small lumen, which small lumens are positioned around one larger, inner lumen. Alternatively, the conductor(s), wound or not, may be positioned between outer insulation 60 and another insulating tube between outer insulation 60 and liner 100. In yet another alternative, the conductor(s), wound or not, may be positioned between outer insulation 60 and liner 100.

In various embodiments, the material of outer insulation 60 is melted and reflowed. For instance, after the tube of outer insulation 60 is assembled, such as described above, the lead assembly may be placed in a fixture and heated, causing the material of outer insulation 60 to flow around conductor winding 40. If liner 100 is in place prior to this process, the material of insulation 60 will be prevented from encroaching into lumen 30, just as is over-mold material 50/52/54. Alternatively, rather than heating and reflowing a tube of outer insulation 60, the lead assembly, or a portion of it, may be placed in a mold, and the material of outer insulation 60 may be injected into the mold.

Because Teflon and the like have such a low coefficient of friction, it is challenging to obtain permanent adhesion with other materials. Methods to improve adhesion to such materials include etching, as mentioned above, and/or plasma treating. In the case of etching, the outer diameter of the liner 100 can be etched in order to increase adhesion to other materials (the material of outer insulation 60 or over-mold material 50/52/54) in the lead. Etching of the inner diameter of the Teflon is not required, thus the ability to slide wires through lumen 30, when lined with Teflon, is not sacrificed.

In some embodiments, liner 100 is a permanent member of the lead. For instance, liner 100 may be located inside the inner diameter of the coil. In other embodiments, liner 100 is removable. For instance, during construction, liner 100 may be inserted between conductor winding 40 and the mandrel, for instance, before or after outer tubing 60 has been assembled onto the lead. When using a removable liner, it may be preferable to refrain from etching or the like of the liner material, thereby easing assembly and removal of the liner.

When using such a lead with removable liner 100, the following is made possible: The lead is first placed over a recording microelectrode. Next, the recording microelectrode is removed. Finally, liner 100 is removed. This design allows for loading of the material of liner 100 with barium sulfate, bismuth subcarbonate, or the like, to increase visibility of the lead (prior to liner removal) under x-ray. These compounds are not commonly used in permanent devices. Using a lead with a removable liner would also decrease the stiffness of the permanently implanted lead.

While the invention herein disclosed has been described by means of specific embodiments and applications thereof, numerous modifications and variations could be made thereto by those skilled in the art without departing from the scope of the invention set forth in the claims.

What is claimed is:

1. A medical lead, comprising:
at least one electrode at a distal end of a lead;
at least one conductor winding defining at least one lumen of the lead and providing electrical connection to the at least one electrode;
insulation along at least a portion of at least the outer surface of the lead; and
at least one liner within the at least one winding, which at least one liner creates at least one lined lumen.

2. The lead of claim 1 further comprising at least one molded portion, where the at least one liner prevents at least some of the at least one molded portion from entering the at least one lumen.

3. The lead of claim 1 wherein the at least one liner is made of polytetrafluoroethylene.

4. The lead of claim 1 wherein the at least one liner is made of at least one of fluorinated ethylene propylene, polyurethane, polyester, and polyimide.

5. The lead of claim 1 wherein a material of the at least one liner and a material of the insulation are the same material.

6. The lead of claim 1 wherein the at least one conductor winding is embedded in the at least one liner.

7. The lead of claim 1 wherein the insulation is reflowed.

8. The lead of claim 1 wherein the at least one conductor winding is embedded in at least one insulating material.

9. The lead of claim 1 further comprising a tube of insulation positioned between at least a portion of the at least one conductor winding and at least a portion of the at least one liner.

10. The lead of claim 1 wherein the at least one liner is permanent.

11. The lead of claim 1 wherein the at least one liner is removable.

12. The lead of claim 1 wherein the conductor winding comprises at least one wound wire.

13. The lead of claim 1 wherein the conductor winding comprises at least one wound cable.

14. The lead of claim 1 wherein at least a portion of an outer surface of the at least one liner is treated to improve adhesion.

15. The lead of claim 1 wherein at least a portion of the at least one liner is loaded with at least one material visible under x-ray.

16. A medical lead, comprising:
at least one electrode at a distal end of a lead;
at least one conductor providing electrical connection to the at least one electrode;
an outer insulation extending from the at least one electrode along at least a portion of at least the outer surface of the lead;
an inner insulation positioned within at least a portion of the outer insulation, which insulation defines at least one lumen within the lead; and
at least one liner positioned within the at least one lumen within the inner insulation, thereby creating a lined lumen.

17. The lead of claim 16 wherein the outer insulation and the inner insulation are one insulation.

18. The lead of claim 16 wherein the at least one conductor is positioned in at least the inner insulation.

19. The lead of claim 16 further comprising at least one molded portion, where the at least one liner prevents at least some of the at least one molded portion from entering the at least one lumen.

20. The lead of claim 16 wherein the at least one liner is made of at least one of polytetrafluoroethylene, fluorinated ethylene propylene, polyurethane, polyester, and polyimide.

21. A method of lead assembly construction, comprising:
providing a mandrel used during construction of a lead assembly;
positioning a liner on the mandrel;
positioning at least one conductor around the liner;
positioning at least one insulating material over the at least one conductor; and
over-molding at least a portion of the lead assembly.

22. The method of claim 21 wherein the liner is made of at least one of polytetrafluoroethylene, fluorinated ethylene propylene, polyurethane, polyester, and polyimide.

23. The method of claim 21 wherein at least a portion of an outer surface of the liner is treated to improve adhesion.

24. The method of claim 21 further comprising reflowing the at least one insulating material.

25. The method of claim 21 wherein the at least one conductor is positioned in at least one conductor insulating material.

26. A medical lead, comprising:
means for delivering electrical stimulation, which delivery means are positioned on a lead;
means connected to the delivery means for conducting electrical signals to the delivery means;
means for insulating at least a portion of at least the outer surface of the lead;
means for insulating the conducting means, which conductor insulating means define at least one lumen;
means for insulating at least the outer surface of the lead, which outer insulating means is positioned along at least a portion of the outer surface of the lead; and
means for lining the at least one lumen, which lining is positioned within the at least one lumen and creates at least one lined lumen.

27. The lead of claim 26 wherein the lining means comprises means for providing a lumen with a coefficient of static friction between steel and the lining means of less than approximately 0.20.

28. The lead of claim 26 wherein at least a portion of an outer surface of the lining means is treated to improve adhesion.

29. The lead of claim 26 wherein the at least one conducting means is positioned in the at least one conductor insulating means.
